(19) United States
(12) Patent Application Publication
(10) Pub. No.: US 2017/0038607 A1
(43) Pub. Date: Feb. 9, 2017

(54) Enhanced-reality electronic device for low-vision pathologies, and implant procedure

(71) Applicant: Rafael Camara, Madrid (ES)

(72) Inventor: Rafael Camara, Madrid (ES)

(21) Appl. No.: 14/817,240

(22) Filed: Aug. 4, 2015

(51) Int. Cl. G02C 11/00; H04N 5/77; H04N 9/31; A61F 9/00; G02C 5/14; G02C 3/00; G02C 7/10; H04L 29/08; H04N 5/225; G02C 7/16

(52) U.S. Cl. CPC G02C 11/10; H04N 5/225; H04N 5/77; H04N 9/3147; G02C 11/12; G02C 7/16; G02C 5/143; G02C 3/003; G02C 7/10; H04L 67/10; A61F 9/00; G02C 2200/02; G02C 2200/04; G02C 2202/10; A61B 90/36

(57) ABSTRACT

An electronic enhanced-reality device for low-vision pathologies and an implant procedure to optimise the residual vision of persons with low-vision pathologies, to enhance or recover certain functionalities and autonomy for daily activities, Internet-assisted in cloud mode and which, using patient data along with other data and ophthalmological metadata (big data), plus the adjustment and adaptation of the image captured by a video camera, allows the residual visual of persons affected by low-vision pathologies to be optimised, and operating in such a way that with the optimal parametrisations selected and loaded on to the device, the cameras switch on automatically and their images replace the previous image pre-loaded in the projection area (10).

These images are processed by the signal processor (9) and projected by the image projector (31) on to the projection areas (10).

These images are the ones which the patient will perceive.

Ophthalmologist-patient interaction will enable the ophthalmologist to place the images in the right place for the patient in the projection area (10), suitably adapting the images from the camera in each eye independently or simultaneously in both, to optimise their residual vision.

### OBJECT OF THE INVENTION

The proposed invention refers to an electronic enhanced-reality device and implant procedure to optimise the residual vision of persons with low-vision pathologies, Internet-assisted in cloud mode and which, using the patient data along with other data and ophthalmological metadata (big data) plus the adjustment and adaptation of the image taken on a video camera, makes it possible to optimise the residual sight of those affected by low vision pathologies, to improve or recover certain functionalities and autonomy for daily activities.

Once configured, the electronic enhanced-reality device concentrates the functionalities most useful in improving the autonomy and quality of life for low-vision patients in a single device.

Thus it represents a step of disruptive evolution compared with existing low-vision aids.

### FIELD OF THE INVENTION

The field of the invention is considered to extend to the optics industry, that of the manufacturers of electronic devices and the underlying architecture and IT infrastructure carrying the “in cloud” service and “big data” management and analysis.

### PRIOR ART

Spanish Patent P201430168, registered on 10 Feb. 2014 under the title “Dispositivo electrónico de Increment del Ángulo de Visión” (Electronic Device to Increase the Angle of Vision) is a remote predecessor of this invention.

According to the Spanish Society of Low-Vision Specialists (SEEBV), Low Vision is “the visual condition suffered by someone with a significant sight loss which does not improve using suitable correction with glasses, contact lenses and even apt medical, pharmacological or surgical treatment, making activities in daily life difficult.

A patient is considered to suffer from low vision when, following optical correction, their visually acuity in the better eye is 0.3 or less, or the visual field less than 20°.

This loss of vision may originate in a variety of illnesses affecting the optic nerve or the retina, seen in losses in the visual centre or the periphery.”

The visual field is defined as the total area in which objects can be seen in lateral vision (peripheral) when the person focuses the eyes at a central point.

The visual field varies from one person to another. Each eye sees approximately 150° on a horizontal plane, rising to 180° when both eyes are superimposed. This figure is about 130′ on the vertical plane, 60° on the horizontal and 70° below.

Low-vision disability may be due to ailments of the central nervous system such as tumours which may damage or put pressure on the visual organs, or cerebral-vascular accidents or a variety of pathologies such as:

ARMD, Glaucoma, Retinitis Pigmentosa, Diabetic Retinopathy, High Blood-Pressure, Multiple Sclerosis, Optic Glioma, Hyperthyroidism, disorders of the pituitary gland, Retinal detachment and Temporal Arteritis.

Many of these conditions are genetic and most can develop into total blindness.

The main cause of Low Vision in the developed world is Age-Related Macular Degeneration (ARMD).

In addition, some pathologies such as ARMD or MEM (Epiretinal Macular Membrane) produce distortion in the image, which ophthalmologists call metamorphopsia, and which involves seeing lines that would be straight in normal conditions with waves. Apart from straight lines, patients also distort the shape of objects or persons they look at.

Recovery of sight in patients who have lost it completely or are at an advanced stage of Low Vision is a dream pursued in various types of research.

Professor Eberhart Zrenner of Tu□bingen University in Germany (http://retina-implant.de/) has tested a prosthesis called “Retina” implanted in a patient's retina, and which has hundreds of micro-photodiodes that receive light from an outside camera and excite the optic nerve, transmitting the image to the brain where it is reproduced.

More recent is the “Argus” system of the United States company Second Sight (http://www.secondsight.com/) which uses a different method but which does likewise require an operation and implant inside the eye. This system was used on a patient in Spain in 2014 at Clinica Barraquer, and there are at present some hundred cases worldwide.

It is seen that there are various prototypes for transmitting the information to the brain via an intermediate processor.

These equipments are complex, requiring delicate operations, and particularly targets patients who, from degenerative evolution or other causes, have lost their sight or are close to doing so.

On the other hand, while this invention does require a prescription from an ophthalmologist/low-vision specialist, it provides the patient with a device which is portable, lightweight, non-invasive (no surgical intervention, injections, etc.) and immediately applicable, targeting the visually impaired to optimise their residual vision by wide-angle format images or with powerful zoom magnification, depending on whether their condition is in the visual field or else in visual acuity.

There is also provision for the device to accept parameterisation to correct visual distortion (metamorphopsia).

This operational flexibility means that this invention is of wide application to a great variety of low-vision pathologies.

During the research and development of this invention we learned of a device named electronic glasses, of the Canadian company eSight, (http://esighteyewear.com/).

According to this company, their glasses are able to enlarge up to 14× and are more effective for patients whose acuity is between 20/60 and 20/400.

It is not suitable for serious tunnel vision, that is where the visual field is reduced.

These are commercial glasses that can be sold to the public without a prescription.

Other known devices available so far, such as Enhanced-Reality Head-Mounted Displays (HMD) or virtual reality (Google Glass, Epson Moverio BT200, Vuzix M100, Sony HMZ-T1, Recon Jet, etc.) are basically for leisure and sporting activities or as a free-hand operational aid.

Developments are known of some computer applications for electronic glasses specifically targeting the “healthcare sector for assistance and remote control, recognition of alterations during surgery, etc”, but in no circumstances with the same dedicated applications as are developed in the invention proposed.

The inventor is unfamiliar with any system which makes it possible, as with this invention, to concentrate the light control required by a Low-Vision patient in a single device, to avoid damaging their delicate retina even further, along with the set of functionalities specific to their pathology and peculiarities.

### DESCRIPTION OF THE INVENTION

The device consists of a electronic eyeglass frame made up of various elements worn by the patient and connected physically or by wifi to a computer terminal where the ophthalmologist/specialist configures the images captured by the device's camera, parameterising different video variables and suitably adapting the camera's images in each of the patient's eyes, independently or at the same time, to optimise their residual vision.

Using the data bases and metadata on the condition, and the patient's characteristics, the image is upgraded at the ophthalmologist/specialist's terminal and simultaneously in the patient's theoretical visual field.

In dialogue with the patient, the ophthalmologist/specialist fine-tunes all the video parameters under consideration, storing all appropriate parametrisations.

Optimal parametrisation is selected, marked and loaded into the device which is configured with as many parametrisations as are considered necessary for the patient's various activities (walking, reading, watching TV, . . . ), so improving their quality of life and independence.

The parametrisations loaded into the device are stored locally and in cloud mode.

The information in the cloud constitutes a data base of the greatest importance for ophthalmological research (big data).

With the device loaded, the patient is able in their daily activity to select at will the customised configuration appropriate to the moment (day/night), the nature of the activity (static/dynamic) and its type (reading, watching TV, etc.),

The ophthalmologist-patient consultation kit will consist at least of the ophthalmologist/specialist's terminal and the enhanced-reality electronic device for the patient with at least BT, Wifi, a high-definition frontal camera and optical zoom, signal processor and projection device to visualise colour and high definition images and to control radiation by attenuating/eliminating any damaging light.

The device's control unit has pushbuttons to command the various parameters—electronic zoom, brightness, contrast, etc.—and contains a rechargeable storage battery for >=8 hours' operation.

There may also be a small battery inside the eyeglass itself.

The device will admit modules for enhanced performance: Internet connection, e-mail, voice control, gesture control, face recognition, inverted colours, infrared camera, plenoptic camera (field light camera) to correct images deformed by metamorphopsia, etc.

The ophthalmologist/specialist's terminal will have at least a touch screen, BT, Wifi, internet browser, e-mail, and the applications and programmes allowing control of the modular functionalities installed in the device.

With the same Operating System, the device and the ophthalmologist/specialist's terminal are interconnected by Wifi or cable so that all controls implemented at the terminal are reproduced, without appreciable latency, in the device.

The ophthalmologist/specialist's terminal can run in local mode to parametrise the device in present/remote situations with the patient, and also connects in cloud mode to access a Big Data repository with Data Mining (collection, extraction, warehousing, analysis, and statistics) for Low Vision pathologies.

Thanks to electronic miniaturisation, a device can be installed in the front-arm of the eyeglass or incorporated into the arms on the eyeglass which basically contains an HD frontal camera to capture images, an image processor and a broadcast system which projects the image in front of the patient.

To make the system flexible and valid for the greatest number of low-vision pathologies the camera, of >=5 megapixels, may have a lens train for optical zoom with focuses ranging from wide-angle, with a field close to 100°, to >=3× optical zoom.

A further possibility is to have a switching double camera, one for wide angle and the other for a powerful zoom.

The electronic device and the ophthalmologist/special terminal each incorporate electronic applications which are interrelated and preloaded in each to accommodate the image taken by the camera to the patient's reduced visual field, optimising their sight.

Device commands (zoom, brightness, contrast, etc.) are managed with pushbuttons incorporated into an external Control Unit connected to the glasses by cable or directly on terminals in the eyeglass.

That Control Unit may also include a rechargeable battery of approximately 8 hours' duration and which can be removed to be replaced by a spare as necessary.

In addition to pushbutton command, the modular HW and SW design allows for the addition of a voice interface for spoken orders: on/off, high/medium/low light level, zoom . . . or other modular interfaces (face recognition, gesture recognition, field light camera, etc).

The device design incorporates a mount with a protective shade in the upper part and on the sides, to protect the eyes from rays entering peripherally.

The electronic device has frames to house graduated lenses/selective filters between the image projected and the patient's eyes. Said frames are fitted by inserting them in two movements at 3 fastening points: two housings on the inside bottom part of the mount and a clip close on the inside upper part of the device's eyeglass.

The device also has other filters for ambient light mounted on the outside front using three magnetic zones to secure them to the mount.

The eyeglass arms are flexible and have grooves for attachment accessories and for better adjustment to the patient's features.

When being display OFF it is possible to see the reality through it (it is translucent), and while operating the projection of the superposed image can be cancelled at any time by pressure, voice control or gesture control, returning instantly to the projection of the images in the same way.

The ophthalmologist/specialist's terminal, using the same operating system as the device will have, in addition to its basic functionalities, internet connection and browsing thru Bluetooth and Wifi connections, preferably with a touch-screen, and will accept the applications and programmes for the modular functionalities the device loads.

This terminal is connected to the device by wifi or cable, making it possible to parametrise the device in its optimal configuration for the patient's condition and characteristics through an optotype or a fixed image previously loaded for easier adaptation at the ophthalmologist/specialist's consultation, or in remote operation with the patient connected directly by the internet through the device or via a Smartphone with a wifi link to the device.

With the optimal parametrisation(s) selected and loaded onto the device, its HD camera connects automatically and its images replace the previous image downloaded during parametrisation.

The computer application loaded into the ophthalmologist/specialist's terminal makes it possible to include patient data, their pathologies and characteristics in exportable files, along with the parametrisations and functionalities loaded into their glasses. Those data are stored locally and transmitted via the internet for integration into a big data global repository in cloud mode.

A patient may, with the parametrisations downloaded, modify the parameters authorised by the ophthalmologist/specialist—zoom, brightness, contrast, etc.—using the pushbuttons on the control unit, or by voice control, to improve their sight for walking, reading, watching TV, attendance at shows, etc.

### DESCRIPTION OF THE DRAWINGS

To complement this description and to aid in a better understanding of the characteristics of the invention, these specifications are accompanied by two sheets of plans, forming an integral part hereof and which, by way of illustration and without limitation, represent the following:

FIG. 1. A diagrammatic front view of the device eyeglass and external parts.

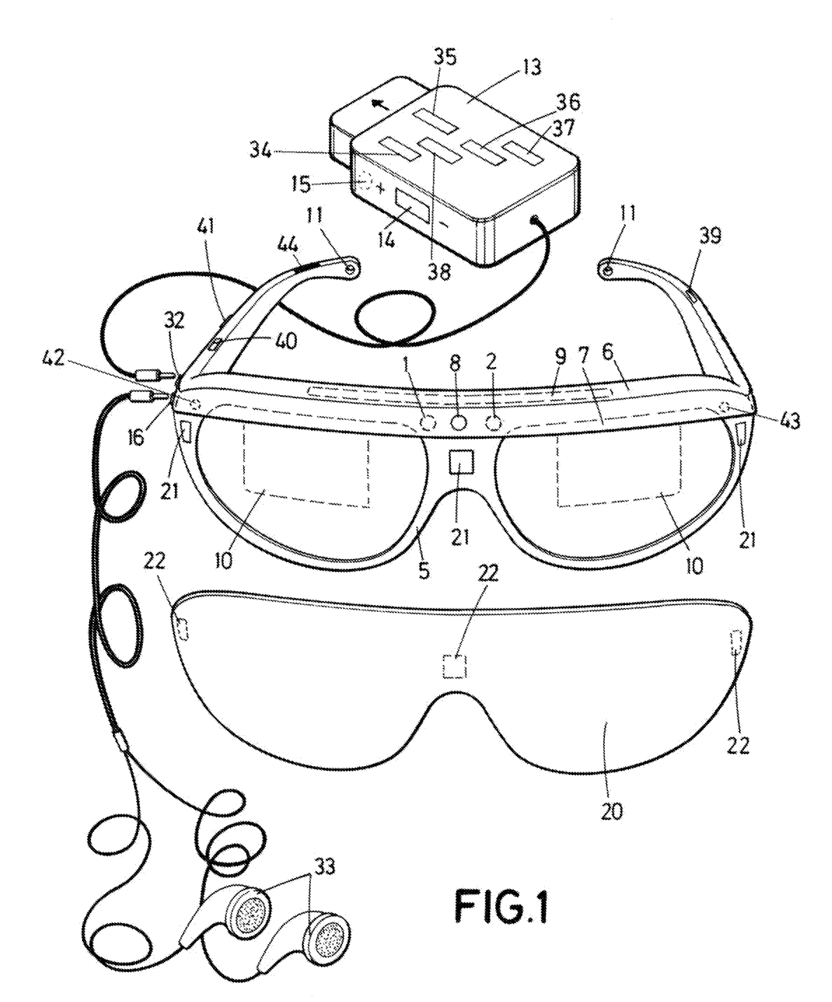

FIG. 2. A diagrammatic rear view of the eyeglass and frame.

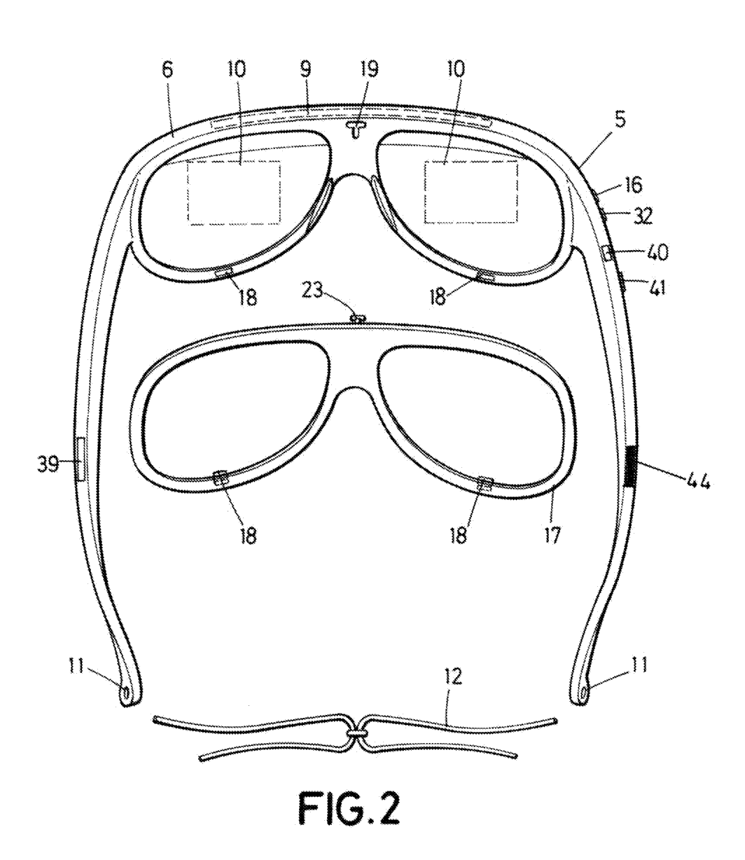

Said figures use a single reference to identify identical elements, among which the following can be made out:

1. Wide-angle camera,

2. Camera with a powerful zoom,

3. The ophthalmologist/specialist's electronic terminal,

4. Cloud sub-system,

5. Glasses frame or mount,

6. Upper sunshade,

7. Side sunshade,

8. HD camera with optical zoom,

9. Signal processor and other HW components,

10. Projection areas,

11. Openings in the arms

12. Safety cord,

13. Control unit,

14. Command pushbuttons,

15. Removable rechargeable battery,

16. Audio output for headphones

17. Frame for graduated lenses/selective filters,

18. Lower inserts to house the frame in the glasses,

19. Click-point to house the frame in the glasses,

20. Filter to attenuate/eliminate external frontal light,

21. Areas to attach the filter to the glasses by magnet,

22. Magnetic attachment zone,

23. Frame mooring click,

31. image projector,

32. Current input connector,

33. External headphones,

34. Voice control,

35. Text reader control,

36. Face recognition,

37. Gesture recognition,

38. Hands-free Smartphone calls and notification,

39. Auxiliary battery,

40. Wi-fi/BT connections,

41. USB takeoff

42. infrared camera,

43. Field light camera,

44. Voice control module with microphone.

45. Distorted image control

### A PREFERENTIAL EMBODIMENT OF THE INVENTION

An electronic enhanced-reality device and implant procedure making it possible to optimise the residual vision of persons with low-vision pathologies, Internet-assisted in cloud mode and which, using the patient data along with other data and ophthalmological metadata (big data), plus adjustment and adaptation of the image taken by a video-camera, makes it possible to optimise the visual residue of those affected by low-vision pathologies, to enhance or recover certain functionalities and autonomy in daily activities.

While this invention does require a prescription from a low-vision ophthalmologist/specialist, and provides the patient with a device which is portable, lightweight, non-invasive (no surgical intervention, injections, etc.) and immediately applicable, targeting the visually impaired to optimise their residual vision by wide-angle format images or with powerful zoom magnification, depending on whether their condition is in the visual field or else related to visual acuity.

There is also provision for the device to accept parameterisation to correct visual distortion (metamorphopsia).

This operational flexibility means that this invention is of wide application to a wide range of low-vision pathologies.

At any time during operation, the device visor may be set simply to translucent, cancelling the image projected in enhanced-reality mode by pressure, voice control or gesture control, returning instantly to the projection of virtual reality images in the same way.

The enhanced-reality electronic device for low-vision pathologies allowing sight to be improved by projecting colour and HD video images in the patient's residual visual area consists of the following hardware and software components:

The hardware comprises the following elements:
a. a eyeglass frame (5) with its adjustment arms made up of at least:
   sunshades above (6) and alongside (7) protecting the patient from external light rays,
   an HD frontal camera (8) with optic zoom to capture video images, alternatively locating four different camera types on the front.
   a camera with a wide-angle device (1) and
   a camera with a powerful zoom device (2)
   an infrared camera (42)
   a field light camera (43) (plenoptic camera)
   a signal processor (9) for the camera used,
   two projection areas (10), one for each eye, located in the zone of vision in front of each eye and each comprising areas in the form of virtual rectangles,
   two image projectors (31) which can be located in the top of the eyeglass frame (5) or in both arms, projecting their image onto the projection areas (10),
   two orifices (11) at the end of the eyeglass arms intended to house securing elements, and which may be formed by a double cord moored in the middle (12) to create a secure attachment and relieve the patient of the weight of the device on the nose,
   an input connector (32) for the external battery (13),
   an input connector (16) for external headphones (33),
   an upper housing (19) on the inside lower part of eyeglass (5) for the upper attachment of the outside frame (17),
   lower housings (18) for the lower attachment of the outside frame (17)
   two magnetised zones (21) to receive the filter's magnetic adherence zones to attenuate/eliminate frontal light (20).
   a USB takeoff (41) in one side of the eyeglass arm,
   a voice control module with microphone on the lower face of the eyeglass arm (44)
   an auxiliary internal battery (39) in Control Unit of outage in the external supply.

As elements comprising the device, but external, the following are made out:
b. A control unit (13) containing the following elements:
   a removable rechargeable battery (15) as power source
   command pushbuttons (14) to turn the device on and off, for audio level control, brightness level, contrast level, and various elements integrated such as:
   miniaturised CPU with BT/Wi-fi connections (40)
   voice control (34),
   text reader control (35),
   face recognition control (36),
   gesture recognition control (37),
   hands-free smartphone calls (38),
   Distorted image control (45)
c. frame (17) to accommodate graduated lenses or selective-absorption optical filters for short-wave, to protect the patient's delicate retina and made up of a casing whose shape and size are similar to the inside of the front of the eyeglass frame (5).

These frames are placed on the eyeglass frame (5) in 2 movements: first inserting them in the 2 housings at the inside bottom part of the casing (18) and then pressing the click in the upper part (19).
d. filters to attenuateleliminate external frontal light (20) to remove direct vision and visualise only the images through the device's camera.

They consist of a surface of a shape similar to the front of the eyeglass frame (5), placed there simply by bringing the three areas of magnetic adherence (22) close to the 3 magnetic zones (21) on the front of the eyeglass frame (5).
e. external headphones (33) directly connected to the device's audio system via the audio output for headphones (16)
f. electronic terminal (3) comprising a tablet, laptop or CPU with a mouse, to be operated by the ophthalmologist.

It will function using the same operating system as the device, connecting with it by bluetooth or wi-fi.

It can also be connected by the USE takeoff (41) installed in the arm of the device (5).

The ophthalmologist's terminal electronic (3) can in turn connect via the internet with a sub-system in the cloud (4).
g. A cloud sub-system (4) where the data base and global metadata generated for ophthalmological research and optimisation of the process by big data analysis are stored and handled.

The ophthalmologist begins the implant procedure at his or her terminal (3) as the device can be parametrised in its optimal configuration for the patient's condition and characteristics using an optotype or a pre-loaded fixed image for easier adaptation in the ophthalmologist/specialist's consultation, parameterising the image for each eye separately or simultaneously, depending on the patient's condition/characteristics.

Once the optimal parametrisations have been selected and loaded in the device, the selected camera connects automatically and its images replace that previous image pre-loaded in the projection area (10).

Those images are processed by the signal processor (9) and projected by the image projector (31) on to the projection areas (10).

These are the images which the patient will perceive.

Ophthalmologist-patient interaction will enable the ophthalmologist to place the images in the right place for the patient in the projection area (10), suitably adapting the images from the camera in each eye independently or in simultaneously, to optimise their residual vision.

The patient will be able, with their parametrisations loaded, to modify the parameters the ophthalmologist/specialist authorises, such as zoom, brightness, contrast or colours, using the command pushbuttons in the control unit (13) to enhance their vision while walking reading, watching TV, attending shows, etc.

The computer application loaded in the ophthalmologist/specialist's terminal makes it possible to integrate patient data into exportable files, with their pathologies and characteristics, along with the parametrisations and functionalities loaded in their device, storing those data locally and transmitting them by the internet for their inclusion in a big data global repository in cloud mode (4), so that the information is fed back globally using all the data entered on each patient of each ophthalmologist.

Having sufficiently described the nature of the invention and its practical implementation, it must be recorded that the specifications indicated above and represented in the attached drawings may be modified in detail provided that this does not alter their fundamental principles defined in the previous paragraphs and summarised in the following claims,

1. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, characterised because the device hardware consists of the following elements:
a. an eyeglass frame (5) with its adjustment arms, comprising at least:
both upper (6) and lateral (7) sunshades protecting the patient from external light rays,
a frontal HD camera (8) with optical zoom to capture video images,
a processor for the signal (9) from the camera used,
two projection zones (10), one for each eye, located in the zone of vision in front of each eye, each comprising areas in the form of virtual rectangles,
two image projectors (31), which can be placed in the upper part of eyeglass frame (5) or in both arms, to project their image on to the projection areas (10),
two orifices (11) at the end of the eyeglass arms,
a component to attach the eyeglass arms (12) and comprising a double cord secured in its middle,
an input connector (32) for the external control unit (13),
an input connector (16) for external headphones (33),
an upper housing (19) inside eyeglass (5) for the upper attachment of the external frame (17),
lower housings (18) for the lower attachment of the external frame (17),
two magnetised zones (21) to accommodate the magnetic adherence zones of the filter for attenuating/eliminating the frontal light (20),
a USB takeoff (41) in one side of the eyeglass arm,
an internal auxiliary battery (39) for external supply outages,
b. Control unit (13) containing the following elements:
a removable rechargeable battery (15) as power supply device,
command pushbuttons (14) to switch the system on and off, control audio level, brightness, contrast, along with a variety of integrated elements such as:
miniaturised CPU with BT/Wi-fi connections (40)
voice control (34),
text reader control (35),
face recognition control (36),
gesture recognition control (37),
hands-free smartphone calls (38),
distorted image control (45)
c. framework (17) to house graduated lenses or optical filters with selective absorption for short-wavelengths, to protect the patient's delicate retina with a structure whose shape and dimensions are similar to the inside eyeglass frame's (5) front.
These frames are placed on the eyeglass frame (5) in 2 movements: first by inserting them in the 2 housings in the lower inside part of the casing (18), then pressing on the click of the upper part (19).
d. filters to attenuate/suppress external frontal light (20) to eliminate direct vision and visualise just the images via the camera.
They are formed by a treated glass surface whose shape is similar to that of the front of the eyeglass frame (5) where they are fitted simply by bringing the three magnetic adherence zones (22) close to the 3 magnetised areas (21) on the front of eyeglass frame (5).
e. external headphones (33) connected directly to the device's audio system via the audio headphone output (16).
f. an electronic terminal (3) comprising preferably a tablet or laptop with touch-screen, to be operated by the ophthalmologist/specialist.
It will run on the same operative system as the device, connecting with it by bluetooth or wi-fi.
It can also be connected via the USB takeoff (41).
The ophthalmologist's electronic terminal (3) is connected via the internet with a cloud sub-system (4).
g. A cloud sub-system (4) for storage and management of the data base and global metadata generated for ophthalmological research and optimisation of the process using big data analysis.

2. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, as set forth in claim 1, characterised because two cameras may also be used alternately to take the images:
a camera with a powerful optical zoom device (2)
a wide-angle camera (1).

3. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, as set forth in claims 1 and 2, characterised because two other types of camera may be used alternately to take the images:
an infrared camera (42)
or a field light camera (plenoptic camera) (43)

4. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, characterised because the implant procedure is begun by the ophthalmologist from their terminal (3) as the device can be parametrised to its optimal configuration for the patient's condition and characteristics using an optotype or fixed image pre-loaded for easier adaptation in the ophthalmologist/specialist's consultation or, with the patient located elsewhere, in remote mode via an Internet connection.
With the optimal parametrisations selected and loaded onto the device, the camera selected switches on automatically and its images replace the previous image pre-loaded in the area of projection (10).
These images are processed by the signal processor (9) and projected by the image projector (31) on to the projection areas (10).
These images are the ones which the patient will perceive.
Ophthalmologist-patient interaction will enable the ophthalmologist to place the images in the right place for the patient in the projection area (10), suitably adapting the images from the camera in each eye independently or in both at the same time, to optimise their residual vision.

5. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, as set forth in claim 3, characterised because the patient may, with their parametrisations loaded, modify the parameters authorised by the ophthalmologist/specialist, such as zoom, brightness, contrast or colours, using the command pushbuttons on the control unit (13).

6. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, as set forth in claims 3 and 4, characterised because the computer application loaded in to the ophthalmologist/specialist's terminal allows patient data to be included in exportable files with their pathologies and characteristics, along with the parametrisations and functionalities loaded into their device.

7. An electronic enhanced-reality device and implant procedure to optimise the residual vision of persons with low-vision pathologies, Internet-assisted in cloud mode and which, using patient data along with other data and ophthalmological metadata (big data), plus the adjustment and adaptation of the image captured by a video camera, allows the residual visual of persons affected by low-vision pathologies to be optimised, as set forth in claims 3, 4 and 5, characterised because said data are stored locally and transmitted via the Internet for integration into a global big data repository in cloud mode (4), so that the information is fed back using all the data entered on each patient of each ophthalmologist.
